(19) United States
(12) Patent Application Publication
(10) Pub. No.: US 2026/0283504 A1
(43) Pub. Date: Sep. 24, 2026

(54) BIOLOGICAL INFORMATION MEASUREMENT DEVICE, BIOLOGICAL INFORMATION MEASUREMENT METHOD, AND RECORDING MEDIUM

(71) Applicant: OMRON HEALTHCARE Co., Ltd., Kyoto (JP)

(72) Inventors: Yotaro NISHIO, Kyoto (JP); Chiaki EBISU, Kyoto (JP); Tatsuya KOBAYASHI, Kyoto (JP); Mika EZOE, Kyoto (JP); Takanobu YAMAUCHI, Kyoto (JP); Yuki SAKAGUCHI, Kyoto (JP); Tetsuho ONO, Kyoto (JP); Kenichi HINUMA, Kyoto (JP); Iwanori NAMBA, Kyoto (JP)

(21) Appl. No.: 19/683,306

(22) Filed: May 20, 2026

Related U.S. Application Data
(63) Continuation of application No. PCT/JP2025/000570, filed on Jan. 9, 2025.

(30) Foreign Application Priority Data
Mar. 13, 2024 (JP) 2024-038978

(51) Int. Cl. A61B 5/11

(52) U.S. Cl. CPC A61B 5/11

(57) ABSTRACT

To realize desirable measurement for a subject. A biological information measurement device includes a main body portion including a placement surface on which a subject can step, a state information acquisition unit that acquires state information of an input device operated by the subject, and a biological information measurement unit that selects a measurement sequence corresponding to the state information to measure biological information of the subject on the placement surface.

### CROSS-REFERENCE TO RELATED APPLICATIONS

This application is the U.S. national stage application filed pursuant to 35 U.S.C. 365(c) and 120 as a continuation of International Patent Application No. PCT/JP2025/000570, filed Jan. 9, 2025, which application claims priority to Japanese Patent Application No. 2024-038978, filed Mar. 13, 2024, which applications are incorporated herein by reference in their entireties.

### TECHNICAL FIELD

The present invention relates to a biological information measurement device, a biological information measurement method, and a recording medium or program.

### BACKGROUND ART

Patent Document 1 (JP 2016-101504 A) and Patent Document 2 (JP 2014-507213 T) disclose devices that acquire biological signals (IPG, BCG, or the like) with a weight and body composition meter type device and output a biological indicator such as vascular elasticity.

### SUMMARY OF INVENTION

### Technical Problem

A general body composition meter includes a load cell and an electrode unit that comes into contact with a human body with which a weight measurement or a bioelectrical impedance measurement is performed, and displays a result related to a body weight or various body compositions. The time spent for such measurements is several seconds. Thus, the measurement result is not largely affected by body motion of the subject, such as movement of the head for a purpose such as checking the measurement progress state displayed at their feet during measurement. On the other hand, when a function of performing ballistocardiogram (BCG) measurement from a load cell and impedance pulse wave (IPG) measurement from an electrode and presenting cardiovascular state information to a user is added to the body composition meter, body motion that is movement of the head or arm during measurement largely affects the measurement result. Thus, as a result, the measurement time may be prolonged, or remeasurement may be required due to a measurement error occurring as a result. For example, when the subject is gripping or operating an input device used for measurement, body motion caused by the subject moving their head or arm occurs during measurement, and measurement accuracy deteriorates.

The present invention has been made in view of the above circumstances, and an object is to realize desirable measurement for the subject.

### Solution to Problem

According to one aspect of the present invention, a biological information measurement device including: a main body portion including a placement surface on which a subject can step; a state information acquisition unit that acquires state information of an input device operated by the subject; and a biological information measurement unit that selects a measurement sequence corresponding to the state information to measure biological information of the subject on the placement surface is provided. In a case in which the subject is operating or gripping the input device, body motion caused by the subject moving their head or arm occurs during measurement. The state of the input device is a factor that affects body motion of the subject, and it is possible to improve measurement accuracy of biological information by measuring the biological information of the subject in accordance with the state of the input device. According to the biological information measurement device, a measurement sequence corresponding to state information of the input device operated by the subject is selected to perform measurement of biological information of the subject. This enables the biological information of the subject to be measured in consideration of the state of the input device that causes body motion, and therefore the measurement accuracy of the biological information is improved. Even in a case in which a state change of the input device occurs in the middle of measurement, the measurement sequence is changed and selected in accordance with state information of the input device, body motion suppression notification is given to the subject, or the measurement item is switched to a measurement item hardly affected by body motion at an early stage. Therefore, measurement of biological information is easily completed, which leads to an improvement in the measurement success rate. In this manner, it is possible to realize desirable measurement for the subject.

The state information includes information indicating whether the input device is installed on the main body portion, and the biological information measurement unit selects the measurement sequence corresponding to information indicating that the input device is installed on the main body portion or information indicating that the input device is not installed on the main body portion to measure the biological information of the subject. A measurement sequence corresponding to information indicating that the input device is installed on the main body portion of the biological information measurement device or information indicating that the input device is not installed on the main body portion of the biological information measurement device is selected to perform measurement of biological information of the subject. By installing the input device in the main body portion of the biological information measurement device, body motion caused by the subject gripping the input device is suppressed. In a case in which the measurement sequence corresponding to the information indicating that the input device is installed on the main body portion of the biological information measurement device is selected, measurement of the biological information of the subject is performed in a state in which body motion is suppressed, and therefore the measurement accuracy is improved. In a case in which the input device is not installed on the main body portion of the biological information measurement device, there is a possibility that body motion occurs by the subject gripping the input device or the subject operating the input device. In a case in which the measurement sequence corresponding to the information indicating that the input device is not installed on the main body portion of the biological information measurement device is selected, remeasurement due to the body motion can be prevented, and therefore the measurement success rate is improved.

The state information includes information indicating whether the input device is installed around the main body portion, and the biological information measurement unit selects the measurement sequence corresponding to information indicating that the input device is installed around the main body portion or information indicating that the input device is not installed around the main body portion to measure the biological information of the subject. A measurement sequence corresponding to information indicating that the input device is installed around the main body portion of the biological information measurement device or information indicating that the input device is not installed around the main body portion of the biological information measurement device is selected to perform measurement of biological information of the subject. By installing the input device around the main body portion of the biological information measurement device, body motion caused by the subject gripping the input device is suppressed. In a case in which the measurement sequence corresponding to the information indicating that the input device is installed around the main body portion of the biological information measurement device is selected, measurement of the biological information of the subject is performed in a state in which body motion is suppressed, and therefore the measurement accuracy is improved. In a case in which the input device is not installed around the main body portion of the biological information measurement device, there is a possibility that body motion occurs by the subject gripping the input device or the subject operating the input device. In a case in which the measurement sequence corresponding to the information indicating that the input device is not installed around the main body portion of the biological information measurement device is selected, remeasurement due to the body motion can be prevented, and therefore the measurement success rate is improved.

The state information includes information indicating whether the subject is gripping the input device, and the biological information measurement unit selects the measurement sequence corresponding to information indicating that the subject is gripping the input device or information indicating that the subject is not gripping the input device to measure the biological information of the subject. A measurement sequence corresponding to information indicating that the subject is gripping the input device or information indicating that the subject is not gripping the input device is selected to perform measurement of biological information of the subject. In a case in which the subject is gripping the input device, there is a possibility that body motion occurs by the subject operating the input device. In a case in which the measurement sequence corresponding to the information indicating that the subject is gripping the input device is selected, remeasurement due to the body motion can be prevented, and therefore the measurement success rate is improved. In a case in which the subject is not gripping the input device, body motion caused by the subject operating the input device is suppressed. In a case in which the measurement sequence corresponding to the information indicating that the subject is not gripping the input device is selected, measurement of the biological information of the subject is performed in a state in which body motion is suppressed, and therefore the measurement accuracy is improved.

The biological information measurement unit selects at least one measurement item of a plurality of measurement items related to a living body of the subject on the placement surface based on the state information to measure the biological information of the subject. For example, in a case in which the state of the input device is a state that leads to the occurrence of body motion, the measurement success rate is improved by selecting a measurement item hardly affected by the body motion. In a case in which a measurement item not easily affected by body motion is selected, no other measurement item is selected, and therefore the measurement time is shortened.

The state information includes information indicating whether the input device is installed on the main body portion, and the number of the measurement items selected in a case in which the state information includes information indicating that the input device is not installed on the main body portion is smaller than the number of the measurement items selected in a case in which the state information includes information indicating that the input device is installed on the main body portion. In the case in which the state information includes information indicating that the input device is not installed on the main body portion, the number of measurement items to be selected is small, and therefore the time from the start to the completion of measurement of the biological information of the subject is shortened.

The state information includes information indicating whether the input device is installed around the main body portion, and the number of the measurement items selected in a case in which the state information includes information indicating that the input device is not installed around the main body portion is smaller than the number of the measurement items selected in a case in which the state information includes information indicating that the input device is installed around the main body portion. In the case in which the state information includes information indicating that the input device is not installed around the main body portion, the number of measurement items to be selected is small, and therefore the time from the start to the completion of measurement of the biological information of the subject is shortened.

The state information includes information indicating whether the subject is gripping the input device, and the number of the measurement items selected in a case in which the state information includes information indicating that the subject is gripping the input device is smaller than the number of the measurement items selected in a case in which the state information includes information indicating that the subject is not gripping the input device. In the case in which the state information includes information indicating that the subject is gripping the input device, the number of measurement items to be selected is small, and therefore the time from the start to the completion of measurement of the biological information of the subject is shortened.

The biological information measurement device includes a body motion measurement unit that measures body motion of the subject on the placement surface, and the biological information measurement unit selects the measurement sequence corresponding to the state information and a measurement result of the body motion measurement unit to measure the biological information of the subject on the placement surface. For example, even in a case in which the state of the input device is a state that leads to the occurrence of the body motion of the subject, there is a case in which the body motion of the subject does not occur. For example, even in a case in which the state of the input device is a state that does not lead to the occurrence of the body motion of the subject, there is a case in which the body motion of the subject occurs. By the body motion measurement unit measuring the body motion of the subject, the state of the subject can be captured more accurately, and the biological information of the subject can be measured more accurately. Since the biological information of the subject can be measured in consideration of the state of the input device and the body motion of the subject, the measurement accuracy of the biological information is improved. Even in a case in which a state change of the input device occurs in the middle of measurement, measurement of biological information is easily completed, which leads to an improvement in the measurement success rate.

The biological information measurement device includes a body motion measurement unit that measures body motion of the subject on the placement surface, and the biological information measurement unit selects at least one measurement item of the plurality of measurement items related to the living body of the subject based on the state information and a measurement result of the body motion measurement unit to measure the biological information of the subject on the placement surface. For example, in a case in which the state of the input device does not lead to the occurrence of body motion but body motion of the subject occurs, the measurement success rate is improved by selecting a measurement item hardly affected by the body motion. In a case in which a measurement item not easily affected by body motion is selected, no other measurement item is selected, and therefore the measurement time is shortened.

Note that the present invention can also be understood as a biological information measurement method in which a computer executes at least part of the above processing, a biological information measurement method including at least part of the above processing, a program for causing a computer to execute at least part of the above processing, or a computer-readable recording medium on which such a program is non-transitorily recorded. Note that the configurations and processes described above can be combined with one another to constitute the present invention unless the combination leads to technical contradiction.

### Advantageous Effects

According to the present invention, it is possible to realize desirable measurement for the subject.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a diagram illustrating a schematic configuration of a biological information measurement device according to an embodiment.

FIG. 2 is a diagram illustrating a configuration of an input device.

FIG. 3 is a diagram illustrating a schematic configuration of a biological information measurement device.

FIG. 4 is a diagram illustrating a schematic configuration of a biological information measurement device.

FIG. 5 is a side view of a biological information measurement device.

FIG. 6 is a diagram illustrating a schematic configuration of a biological information measurement device.

FIG. 7 is a side view of a biological information measurement device.

FIG. 8 is a diagram illustrating a schematic configuration of a biological information measurement device.

FIG. 9 is a side view of a biological information measurement device.

FIG. 10 is a block diagram illustrating a configuration of a biological information measurement device according to an embodiment.

FIG. 11 is a diagram illustrating an arrangement of a plurality of load cells.

FIG. 12 is a diagram illustrating an arrangement of a plurality of load cells and an acceleration sensor.

FIG. 13 is a block diagram illustrating a functional configuration of a control unit.

FIG. 14 is a diagram describing a relationship among a BCG waveform, a foot IPG waveform, and a PTT.

FIG. 15 is a flowchart for describing an operation of a biological information measurement device.

### DESCRIPTION OF EMBODIMENTS

Embodiments will be described below with reference to the drawings. The embodiment described below is one aspect of the present application and does not limit the scope of the present application.

FIG. 1 is a diagram illustrating a schematic configuration of a biological information measurement device 1 according to the present embodiment. A biological information measurement device 1 illustrated in FIG. 1 includes a main body portion (housing) 2 including a front surface (placement surface) on which a subject such as a user can step, a display unit 3, current applying electrodes 4 and 5, and voltage measuring electrodes 6 and 7.

The current applying electrode 4 and the voltage measuring electrode 6 are arranged on the front surface of the main body portion 2 such that the front end side (toe side) of the bottom surface of the left foot is in contact with the current applying electrode 4 and the rear end side (heel side) of the bottom surface of the left foot is in contact with the voltage measuring electrode 6 in a case in which a subject such as a user steps on the front surface of the main body portion 2. The current applying electrode 5 and the voltage measuring electrode 7 are arranged on the front surface of the main body portion 2 such that the front end side (toe side) of the bottom surface of the right foot is in contact with the current applying electrode 5 and the rear end side (heel side) of the bottom surface of the right foot is in contact with the voltage measuring electrode 7 in a case in which the subject steps on the front surface of the main body portion 2. The subject steps on the front surface of the biological information measurement device 1 such that the front end side and the rear end side of the bottom surface of the left foot of the subject are in contact with the current applying electrode 4 and the voltage measuring electrode 6, respectively, and the front end side and the rear end side of the bottom surface of the right foot of the subject are in contact with the current applying electrode 5 and the voltage measuring electrode 7, respectively. The arrangement of the current applying electrode 4 and the voltage measuring electrode 6 may be reversed, or the arrangement of the current applying electrode 5 and the voltage measuring electrode 7 may be reversed.

The display unit 3 is provided on the bottom surface of a recess portion 8 formed on the front surface of the main body portion 2. The recess portion 8 is formed in a central portion of the front surface of the main body portion 2. The recess portion 8 is a housing portion (holder) recessed from the front surface of the main body portion 2. As the display unit 3, for example, a liquid crystal display panel such as a liquid crystal display (LCD) or an electroluminescence (EL) display is adopted. The display unit 3 displays measurement results, various types of information, and data. By housing the input device operated by the subject in the recess portion 8 of the main body portion 2, the input device can be installed on the main body portion 2. The input device is an operation unit for receiving an operation from the subject and inputting information or data to the biological information measurement device 1. The input device may be a dedicated device attached to the biological information measurement device 1 or an information processing terminal owned by the subject. The information processing terminal is, for example, a mobile terminal, a smartphone, a tablet terminal, or the like.

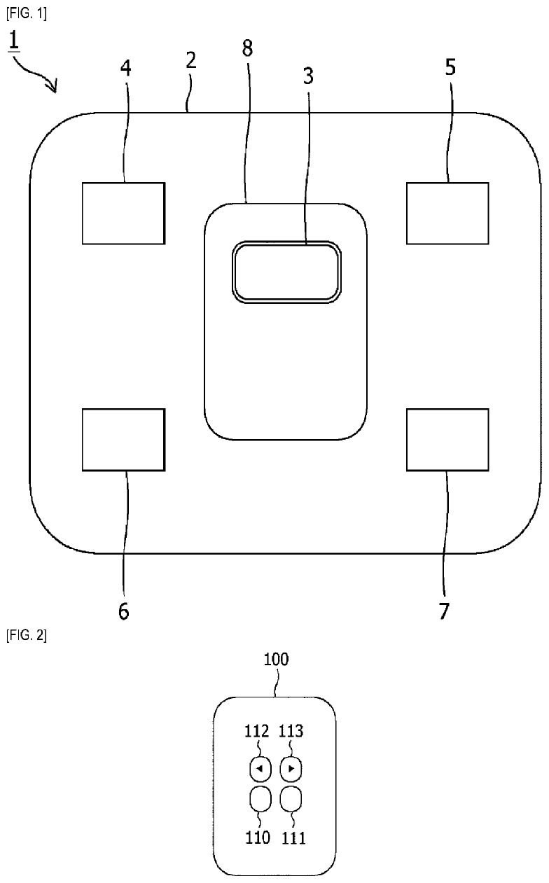

FIG. 2 is a diagram illustrating the configuration of an input device 100. The input device 100 illustrated in FIG. 2 is a dedicated device attached to the biological information measurement device 1. The input device 100 illustrated in FIG. 2 includes switches 110 to 113. The switch 110 is a switch that receives input of an instruction to turn the power on or off the biological information measurement device 1 and an instruction to start or end measurement. The switch 111 is a switch that receives an instruction to display various data stored in the biological information measurement device 1 onto the display unit 3. The switches 112 and 113 are switches that receive selection and the like of various data displayed on the display unit 3. The switches 111 to 113 are used to receive data input. The input device 100 illustrated in FIG. 2 may include a display unit that displays information or data.

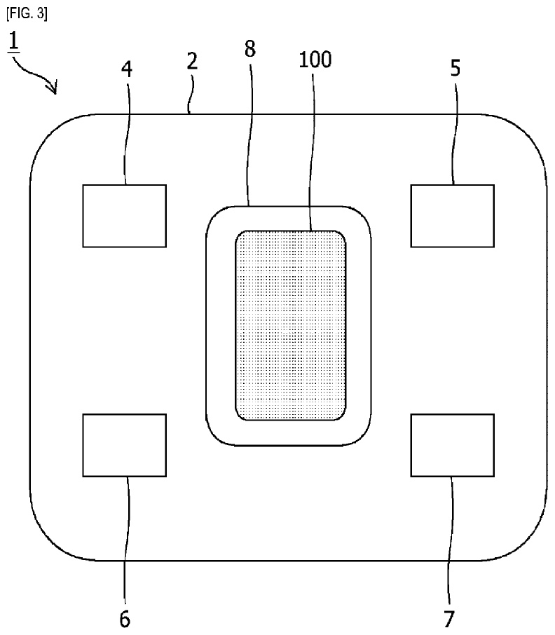

FIG. 3 is a diagram illustrating a schematic configuration of the biological information measurement device 1. In FIG. 3, the input device 100 is installed on the main body portion 2 by housing the input device 100 in the recess portion 8 of the main body portion 2. In FIG. 3, the input device 100 is installed in parallel to the main body portion 2, and the input device 100 is placed flat on the main body portion 2. The display unit 3 is covered by the input device 100 by housing the input device 100 in the recess portion 8 of the main body portion 2. In a case in which the input device 100 is installed on the main body portion 2, the biological information measurement device 1 may shift to a sound mode in which information is conveyed to the subject by sound. In the case in which the input device 100 is installed on the main body portion 2, calibration in weight measurement is performed, and the weight of the input device 100 is excluded from the measurement of the weight of the subject. The biological information measurement device 1 illustrated in FIG. 3 has a structure in which the display unit 3 is hidden when the input device 100 is installed on the main body portion 2. The subject on the front surface of the main body portion 2 cannot visually recognize the display unit 3. Therefore, while measurement of the biological information of the subject is being performed, the motion of the subject viewing the display unit 3 such as by moving their head is suppressed, and the body motion of the subject can be suppressed. This improves the measurement accuracy of the biological information of the subject. FIG. 3 illustrates a configuration example in which the input device 100 is housed in the recess portion 8 of the main body portion 2, but the present invention is not limited to this configuration example. The upper surface of the main body portion 2 may be provided with an edge shape (frame shape), and the input device 100 may be installed inside the frame shape. That is, as a housing portion of the input device 100, the upper surface of the main body portion 2 may be provided with a protruding rib shape that guides the periphery of the input device 100.

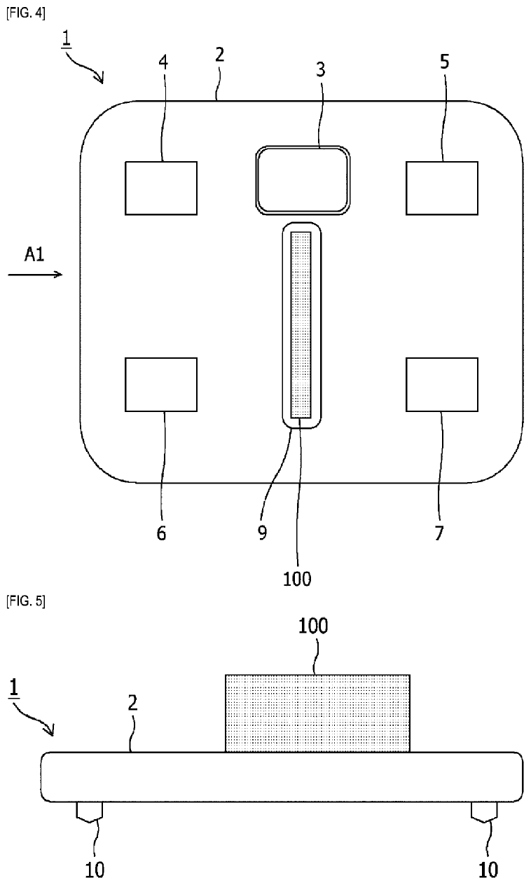

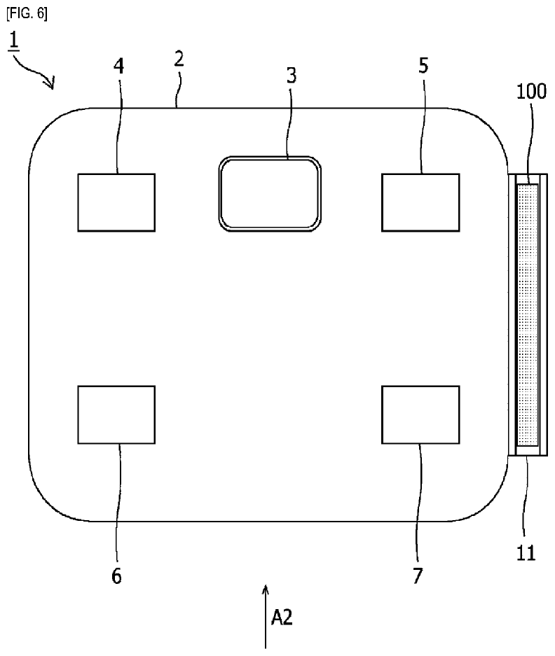

FIG. 4 is a diagram illustrating a schematic configuration of the biological information measurement device 1. The biological information measurement device 1 illustrated in FIG. 4 may be adopted. The biological information measurement device 1 illustrated in FIG. 4 includes the main body portion 2, the display unit 3, the current applying electrodes 4 and 5, and the voltage measuring electrodes 6 and 7. The front surface of the main body portion 2 is provided with the display unit 3. The display unit 3 is arranged between the current applying electrodes 4 and 5. A recess portion 9 is formed in a central portion of the front surface of the main body portion 2. The recess portion 9 is a housing portion (holder) recessed from the front surface of the main body portion 2. In FIG. 4, the input device 100 is installed on the main body portion 2 by housing a part of the input device 100 in the recess portion 9 of the main body portion 2. In the case in which the input device 100 is installed on the main body portion 2, the biological information measurement device 1 may shift to the sound mode in which information is conveyed to the subject by sound. In the case in which the input device 100 is installed on the main body portion 2, calibration in weight measurement is performed, and the weight of the input device 100 is excluded from the measurement of the weight of the subject. In FIG. 4, the input device 100 is installed perpendicularly on the main body portion 2, and the input device 100 is vertically placed on the main body portion 2. FIG. 5 is a side view of the biological information measurement device 1 in a case of being viewed from a direction of arrow A1 of FIG. 4. The rear surface of the main body portion 2 is provided with a plurality of legs 10.

FIG. 6 is a diagram illustrating a schematic configuration of the biological information measurement device 1. The biological information measurement device 1 illustrated in FIG. 6 may be adopted. The biological information measurement device 1 illustrated in FIG. 6 includes the main body portion 2, the display unit 3, the current applying electrodes 4 and 5, and the voltage measuring electrodes 6 and 7. The front surface of the main body portion 2 is provided with the display unit 3. The display unit 3 is arranged between the current applying electrodes 4 and 5. The biological information measurement device 1 illustrated in FIG. 6 includes a housing portion (holder) 11 detachable from the main body portion 2. The housing portion 11 can be attached to an arbitrary position of the main body portion 2. In FIG. 6, the input device 100 is installed on the main body portion 2 by housing a part of the input device 100 in the housing portion 11. In the case in which the input device 100 is installed on the main body portion 2, the biological information measurement device 1 may shift to the sound mode in which information is conveyed to the subject by sound. In the case in which the input device 100 is installed on the main body portion 2, calibration in weight measurement is performed, and the weight of the input device 100 is excluded from the measurement of the weight of the subject. FIG. 7 is a side view of the biological information measurement device 1 in a case of being viewed from a direction of arrow A2 of FIG. 6. The rear surface of the main body portion 2 is provided with the plurality of legs 10.

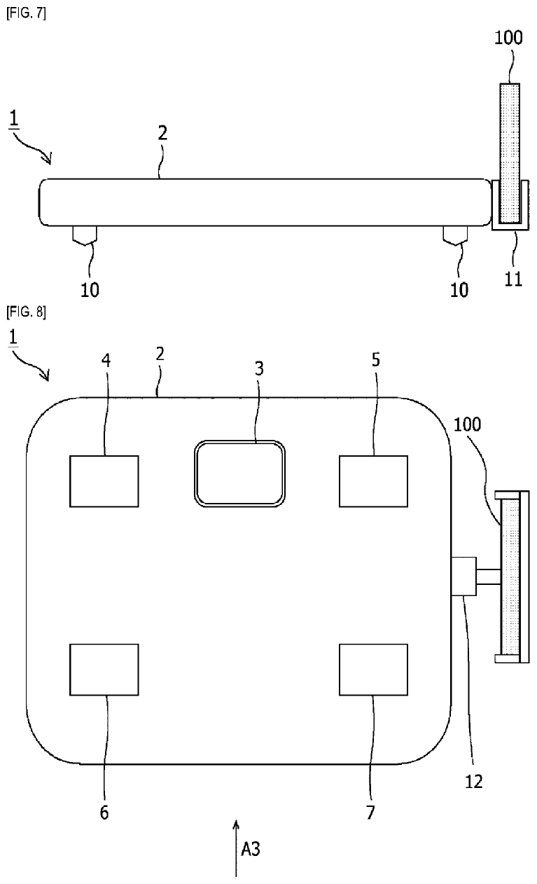

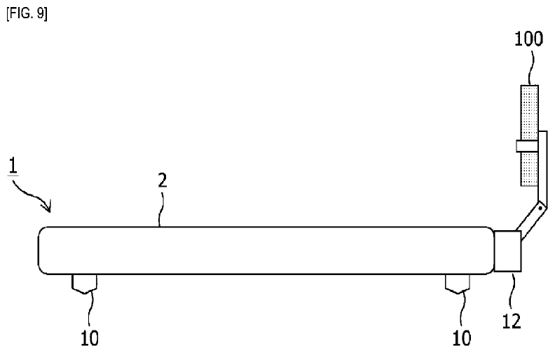

FIG. 8 is a diagram illustrating a schematic configuration of the biological information measurement device 1. The biological information measurement device 1 illustrated in FIG. 8 may be adopted. The biological information measurement device 1 illustrated in FIG. 8 includes the main body portion 2, the display unit 3, the current applying electrodes 4 and 5, and the voltage measuring electrodes 6 and 7. The biological information measurement device 1 illustrated in FIG. 8 includes a support portion (stand) 12 detachable from the main body portion 2. A support portion 12 can be attached to an arbitrary position of the main body portion 2. In FIG. 8, the input device 100 is installed on the main body portion 2 by supporting the input device 100 by the support portion 12. In the case in which the input device 100 is installed on the main body portion 2, the biological information measurement device 1 may shift to the sound mode in which information is conveyed to the subject by sound. In the case in which the input device 100 is installed on the main body portion 2, calibration in weight measurement is performed, and the weight of the input device 100 is excluded from the measurement of the weight of the subject. FIG. 9 is a side view of the biological information measurement device 1 in a case of being viewed from a direction of arrow A3 of FIG. 8. The rear surface of the main body portion 2 is provided with the plurality of legs 10.

In the biological information measurement device 1 illustrated in FIGS. 4, 6, and 8, when the input device 100 is installed on the main body portion 2, the display unit 3 is turned off, and information or data are not displayed on the display unit 3. When measurement of the biological information of the subject is completed, information or data are displayed on the display unit 3. Therefore, while measurement of the biological information of the subject is being performed, the display unit 3 does not display information or data. This suppresses the motion of the subject viewing the display unit 3 such as by moving their head, and can suppress the body motion of the subject. The subject on the front surface of the main body portion 2 cannot visually recognize the front surface and the rear surface of the input device 100 installed on the main body portion 2. Therefore, while measurement of the biological information of the subject is being performed, the motion of the subject viewing the input device 100 such as by moving their head is suppressed, and the body motion of the subject can be suppressed. This improves the measurement accuracy of the biological information of the subject.

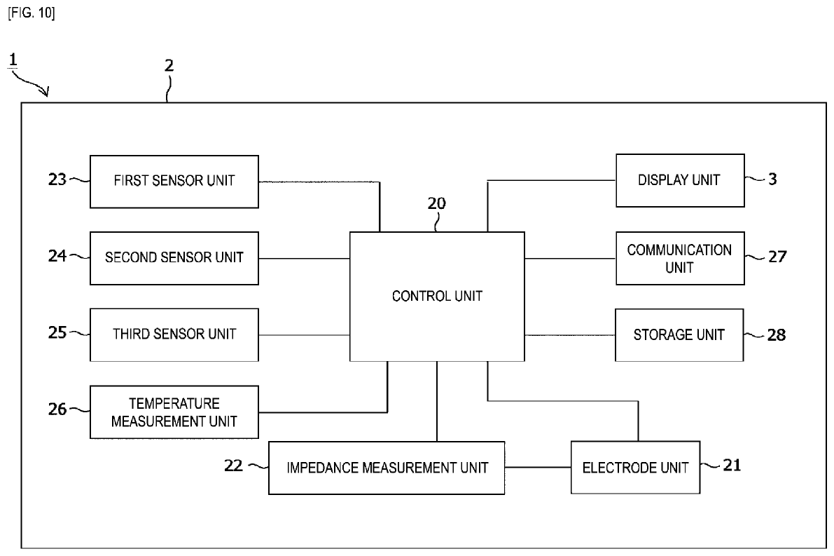

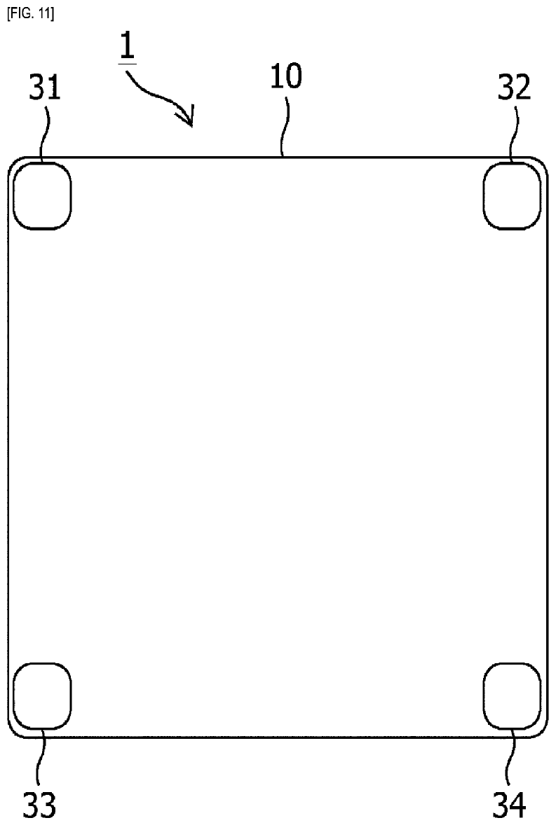

FIG. 10 is a block diagram illustrating the configuration of the biological information measurement device 1 according to an embodiment. The biological information measurement device 1 includes the display unit 3, a control unit 20, an electrode unit 21, an impedance measurement unit 22, a first sensor unit 23, a second sensor unit 24, a third sensor unit 25, a temperature measurement unit 26, a communication unit 27, and a storage unit 28.

The control unit 20 is a control device (controller) that controls the entire operation of the biological information measurement device 1. The control unit 20 may be configured by a dedicated device or may be configured by a general-purpose computer. The control unit 20 includes hardware resources such as a central processing unit (CPU), a memory, and a storage. The memory may be a random access memory (RAM). The storage may be a non-volatile storage device such as a read only memory (ROM) or a flash memory. The functions of the control unit 20 as the processing units (functional units) are realized by loading a program stored in the storage onto a memory and executing the program by a processor. The configuration of the control unit 20 is not limited to the above. For example, the functions of the control unit 20 may be entirely or partially configured by a circuit such as an ASIC or an FPGA, or the functions of the control unit 20 may be entirely or partially executed by a cloud server or another device.

The electrode unit 21 includes the current applying electrodes 4 and 5 for applying a constant current to the body of the subject in order to measure the bioelectrical impedance of the subject, and the voltage measuring electrodes 6 and 7 for measuring a voltage at the time of current application. The electrode unit 21 may include other electrodes different from the current applying electrodes 4 and 5 and the voltage measuring electrodes 6 and 7.

The impedance measurement unit 22 measures the bioelectrical impedance of the subject based on each value of the current applied to the body of the subject from the current applying electrodes 4 and 5 and the voltage measured by the voltage measuring electrodes 6 and 7. The impedance measurement unit 22 outputs the bioelectrical impedance of the subject to the control unit 20. The impedance measurement unit 22 may output the bioelectrical impedance of the subject as an impedance pulse wave.

The first sensor unit 23 includes a photoelectric sensor. The photoelectric sensor includes a light emitting diode (LED) that is a light emission unit that generates light and a photodiode that is a light reception unit that receives light. The first sensor unit 23 outputs the measurement value measured by the photoelectric sensor to the control unit 20. The second sensor unit 24 has a plurality of load cells (strain gauges). The second sensor unit 24 outputs the measurement values measured by the plurality of load cells to the control unit 20. A cushioning material such as a sponge may be provided inside the main body portion 2. This avoids the measurement values measured by the plurality of load cells from being affected in a case in which the input device 100 vibrates.

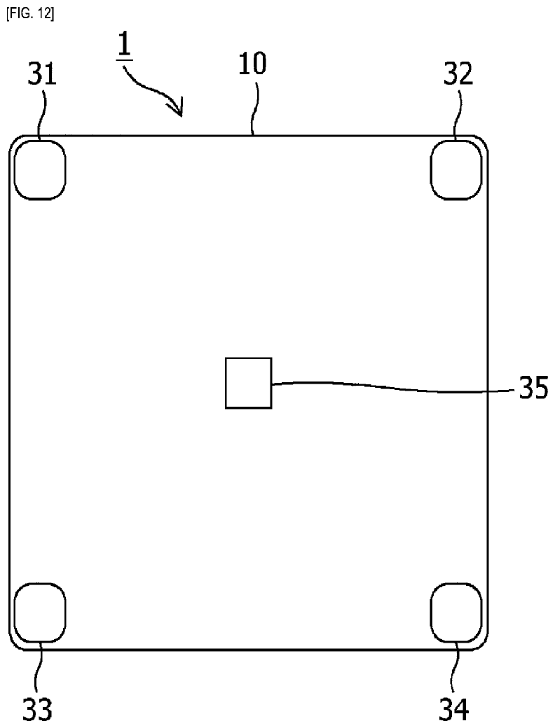

FIG. 11 is a diagram illustrating an arrangement of the plurality of load cells. As illustrated in FIG. 11, load cells 31 to 34 are arranged on the rear surface (back surface) side of the main body portion 2. The load cells 31 to 34 are arranged at the four corners, respectively, on the rear surface side of the main body portion 2. The second sensor unit 24 may include an acceleration sensor. The second sensor unit 24 may output the measurement value measured by the acceleration sensor to the control unit 20.

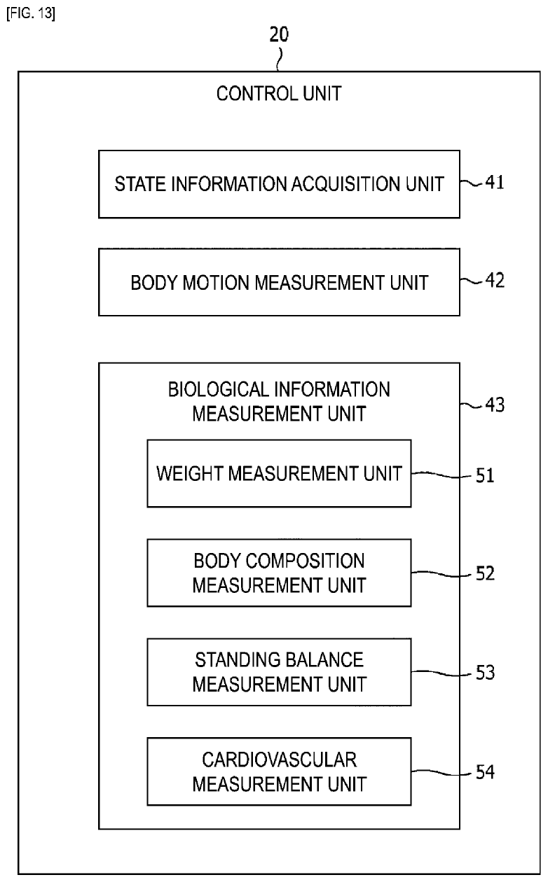

FIG. 12 is a diagram illustrating an arrangement of the plurality of load cells and the acceleration sensor. As illustrated in FIG. 12, the load cells 31 to 34 and an acceleration sensor 35 are arranged on the rear surface side of the main body portion 2. The load cells 31 to 34 are arranged at the four corners, respectively, on the rear surface side of the main body portion 2. The acceleration sensor 35 is arranged in a central portion on the rear surface side of the main body portion 2.

The third sensor unit 25 includes a sound-collecting microphone. The third sensor unit 25 outputs the measurement value measured by the sound-collecting microphone to the control unit 20. The temperature measurement unit 26 measures the temperature of the sole of the foot of the subject on the front surface of the main body portion 2. The temperature measurement unit 26 includes a temperature sensor. The temperature sensor is arranged on the front surface of the main body portion 2. The temperature sensor may be arranged near the current applying electrodes 4 and 5, or may be arranged near the voltage measuring electrodes 6 and 7. The communication unit 27 is an interface for performing wired or wireless communication. The communication unit 27 performs communication with the input device 100.

The storage unit 28 stores information and data. The storage unit 28 may include at least one of a memory such as a RAM and a storage. The storage may be a non-volatile storage device such as a ROM or a flash memory. The storage unit 28 stores pieces of information such as personal data of the subject and measurement results of the biological information measurement device 1. The personal data is used when calculating the body composition of the subject. The personal data includes at least the height and weight of the subject, and may further include the age and gender of the subject, as well as other types of information. By the subject stepping on the front surface of the main body portion 2, the weight of the subject is measured. By the subject operating the input device 100, the height, age, sex, and other information of the subject are input to the biological information measurement device 1 and stored in the storage unit 28. In a case in which the height, age, sex, and other information of the subject are registered in the input device 100, the control unit 20 may acquire and store, in the storage unit 28, the height, age, sex, and other information of the subject from the input device 100.

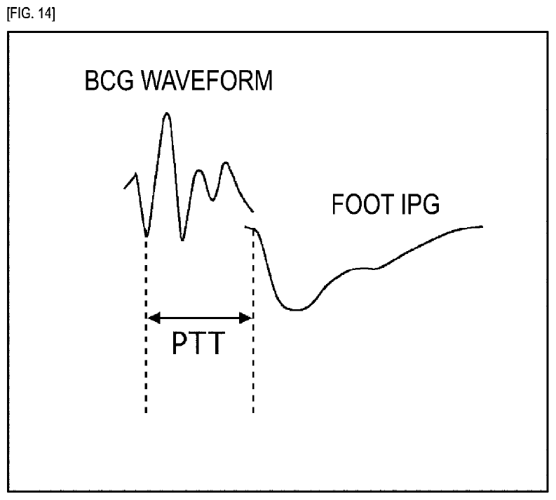

FIG. 13 is a block diagram illustrating a functional configuration of the control unit 20. The control unit 20 includes a state information acquisition unit 41, a body motion measurement unit 42, and a biological information measurement unit 43. The biological information measurement unit 43 includes a weight measurement unit 51, a body composition measurement unit 52, a standing balance measurement unit 53, and a cardiovascular measurement unit 54.

The state information acquisition unit 41 acquires information (state information) regarding the state of the input device 100. The body motion measurement unit 42 measures the body motion of the subject on the front surface of the biological information measurement device 1 based on the measurement value output from at least one of the first sensor unit 23, the second sensor unit 24, and the electrode unit 21. The weight measurement unit 51 measures the weight of the subject on the front surface of the biological information measurement device 1 based on the measurement value output from the second sensor unit 24.

The body composition measurement unit 52 measures the body composition of the subject on the front surface of the biological information measurement device 1 based on the personal data of the subject, the bioelectrical impedance output from the impedance measurement unit 22, and the weight of the subject in accordance with a predetermined algorithm. The body composition is an indicator indicating a percentage or an amount of tissue that constitutes the body. Examples of the body composition include, but are not limited to, body fat percentage, visceral fat level, skeletal muscle percentage, basal metabolism, body age, BMI, muscle percentage, muscle mass, body fat mass, bone mass, water content, and the like. The personal data of the subject includes the weight and the bioelectrical impedance measured in the past. The body composition measurement unit 52 may specify the subject by comparing the weight and the bioelectrical impedance measured in the past with the weight and the bioelectrical impedance measured this time. The body composition measurement unit 52 acquires the personal data of the specified subject from the storage unit 28.

The standing balance measurement unit 53 measures the balance (standing balance) during standing of the subject on the front surface of the biological information measurement device 1 based on the measurement value output from the second sensor unit 24. The standing balance measurement unit 53 may measure the standing balance of the subject by calculating the position of the center of gravity during standing of the subject. The standing balance measurement unit 53 may measure the standing balance of the subject in accordance with a predetermined algorithm.

The cardiovascular measurement unit 54 measures the cardiovascular state of the subject on the front surface of the biological information measurement device 1 based on the measurement value output from the second sensor unit 24 and the bioelectrical impedance output from the impedance measurement unit 22 in accordance with a predetermined algorithm. The cardiovascular measurement unit 54 measures a ballistocardiogram (BCG), which is a vibration in which blood is pumped out of the heart, based on the measurement value output from the second sensor unit 24. The cardiovascular measurement unit 54 measures a leg impedance pulse wave (IPG) and a foot impedance pulse wave (IPG) based on the bioelectrical impedance output from the impedance measurement unit 22. The leg IPG is an indicator indicating a change in the amount of blood in a blood vessel of the leg. The foot IPG is an indicator indicating a change in the amount of blood in a blood vessel of the foot. The cardiovascular measurement unit 54 creates a mean waveform (foot IPG waveform) of the foot IPG with reference to the leg IPG. The cardiovascular measurement unit 54 calculates pulse transit time (PTT) from a BCG waveform and a feature point of the foot IPG waveform.

FIG. 14 is a diagram illustrating a relationship among a BCG waveform, a foot IPG waveform, and a PTT. As illustrated in FIG. 14, the pulse transit time (PTT) can be calculated by taking a difference (time) between a reference point of the BCG waveform and the feature point of the foot IPG waveform. The cardiovascular measurement unit 54 calculates a pulse wave velocity (PWV) by dividing the measurement section distance by the PTT. Since the value of the PWV increases as the blood vessel becomes harder, the PWV is used as an indicator indicating the hardness of the blood vessel. The cardiovascular measurement unit 54 converts the pulse wave velocity (PWV) into an indicator (cardiovascular indicator) representing the heart function or the state of the blood vessel based on a predetermined algorithm, and calculates the cardiovascular indicator. In order to measure the cardiovascular state of the subject, it is necessary to maintain for about 20 to 30 seconds, a state in which there is no body motion of the subject or a state in which the body motion of the subject is small. Therefore, in a case of a state in which there is no body motion of the subject or a state in which the body motion of the subject is small, the cardiovascular measurement unit 54 starts measurement of the cardiovascular state of the subject.

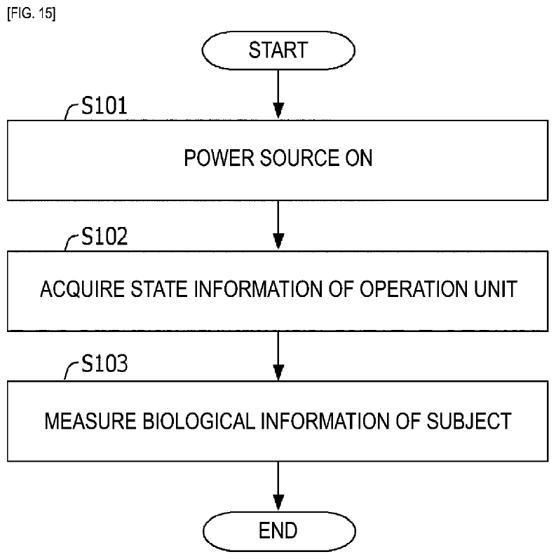

The operation of the biological information measurement device 1 will be described with reference to FIG. 15. FIG. 15 is a flowchart for describing the operation of the biological information measurement device 1. For example, the control unit 20 reads a program stored in the storage unit 28, whereby each process of the flowchart of FIG. 15 is executed.

In step S101, by pressing the switch 110 of the input device 100, the subject turns on the biological information measurement device 1. The biological information measurement device 1 may be turned on by the subject stepping on the front surface of the main body portion 2 in step S101.

In step S102, the state information acquisition unit 41 acquires state information of the input device 100. The state information of the input device 100 will be described. The state information of the input device 100 may include information indicating whether the input device 100 is installed on the main body portion 2.

A process of determining whether the input device 100 is installed on the main body portion 2 will be described. The input device 100 may include a display unit such as a liquid crystal display panel or an EL display, and may output light having a predetermined amplitude or frequency. The state information acquisition unit 41 performs communication with the input device 100 via the communication unit 27, and causes a screen on the display unit of the input device 100 to emit light. The state information acquisition unit 41 may determine whether the input device 100 is installed on the main body portion 2 based on the measurement value output from the first sensor unit 23. The photoelectric sensor of the first sensor unit 23 may be arranged near the installation location of the input device 100. In a case in which light having the predetermined amplitude or frequency is detected, the state information acquisition unit 41 determines that the input device 100 is installed on the main body portion 2. In this case, the state information of the input device 100 includes information indicating that the input device 100 is installed on the main body portion 2. In a case in which light having the predetermined amplitude or frequency is not detected, the state information acquisition unit 41 determines that the input device 100 is not installed on the main body portion 2. In this case, the state information of the input device 100 includes information indicating that the input device 100 is not installed on the main body portion 2.

The input device 100 may include a vibrator constituted by a small motor or the like, and may generate vibration in a predetermined pattern. The state information acquisition unit 41 performs communication with the input device 100 via the communication unit 27, and causes the input device 100 to vibrate. The state information acquisition unit 41 may determine whether the input device 100 is installed on the main body portion 2 based on the measurement value output from the second sensor unit 24. In a case in which vibration of the input device 100 is detected, the state information acquisition unit 41 determines that the input device 100 is installed on the main body portion 2. In a case in which vibration of the input device 100 is not detected, the state information acquisition unit 41 determines that the input device 100 is not installed on the main body portion 2.

The input device 100 may include a sound output device, and may output sound (audible sound or inaudible sound) having a predetermined amplitude or more. The state information acquisition unit 41 performs communication with the input device 100 via the communication unit 27, and causes the input device 100 to output sound having a predetermined amplitude or more. The state information acquisition unit 41 may determine whether the input device 100 is installed on the main body portion 2 based on the measurement value output from the third sensor unit 25. In a case in which sound having a predetermined amplitude or more is detected, the state information acquisition unit 41 determines that the input device 100 is installed on the main body portion 2. In a case in which sound having a predetermined amplitude or more is not detected, the state information acquisition unit 41 determines that the input device 100 is not installed on the main body portion 2.

The input device 100 may include an angular velocity sensor such as a gyro sensor. The state information acquisition unit 41 may perform communication with the input device 100 via communication unit 27, and acquire angle information (measurement value of the angular velocity sensor) of the input device 100. The state information acquisition unit 41 may determine whether the input device 100 is installed on the main body portion 2 based on the measurement value of the angular velocity sensor of the input device 100. In a case in which the measurement value of the angular velocity sensor of the input device 100 falls within a predetermined range, the state information acquisition unit 41 determines that the input device 100 is installed on the main body portion 2. In a case in which the measurement value of the angular velocity sensor of the input device 100 does not fall within the predetermined range, the state information acquisition unit 41 determines that the input device 100 is not installed on the main body portion 2. For example, in the case of FIG. 3, the measurement value of the angular velocity sensor of the input device 100 falls within the predetermined range.

The state information acquisition unit 41 may perform RFID communication with the input device 100 via the communication unit 27, and determine whether an RFID signal can be acquired from the input device 100. In a case in which the RFID signal is acquired from the input device 100, the state information acquisition unit 41 determines that the input device 100 is installed on the main body portion 2. In a case in which the RFID signal is not acquired from the input device 100, it is determined that the input device 100 is not installed on the main body portion 2.

Capacitive detection sensors may be arranged in the recess portions 8 and 9, the housing portion 11, and the support portion 12. The state information acquisition unit 41 may determine whether the input device 100 is installed on the main body portion 2 based on the measurement value (capacitance) of the capacitive detection sensor. In a case in which the capacitance changes, the state information acquisition unit 41 determines that the input device 100 is installed on the main body portion 2. In a case in which the capacitance does not change, the state information acquisition unit 41 determines that the input device 100 is not installed on the main body portion 2.

The state information of the input device 100 may include information indicating whether the input device 100 is installed around the main body portion 2. The process of determining whether the input device 100 is installed around the main body portion 2 will be described. The periphery of the main body portion 2 is, but is not limited to, for example, a wash basin, a shelf, or the like.

The input device 100 may include an acceleration sensor. The state information acquisition unit 41 may perform communication with the input device 100 via the communication unit 27, and acquire acceleration information (measurement value of the acceleration sensor) of the input device 100. The state information acquisition unit 41 may determine whether the input device 100 is installed around the main body portion 2 based on the measurement value of the acceleration sensor of the input device 100. In a case in which the measurement value of the acceleration sensor of the input device 100 is less than a threshold continuously for a predetermined time, the state information acquisition unit 41 determines that the input device 100 is installed around the main body portion 2. In this case, the state information of the input device 100 includes information indicating that the input device 100 is installed around the main body portion 2. In a case in which the input device 100 is installed around the main body portion 2, the input device 100 is not moving and the input device 100 maintains a stable state, and therefore the measurement value of the acceleration sensor of the input device 100 is less than the threshold continuously for the predetermined time. In a case in which the measurement value of the acceleration sensor of the input device 100 is the threshold or more continuously for the predetermined time, the state information acquisition unit 41 determines that the input device 100 is not installed around the main body portion 2. In this case, the state information of the input device 100 includes information indicating that the input device 100 is not installed around the main body portion 2. In a case in which the subject wears the input device 100, the input device 100 is moving and the input device 100 does not maintain a stable state, and therefore the measurement value of the acceleration sensor of the input device 100 is the threshold or more continuously for the predetermined time.

The input device 100 may include a proximity sensor. The state information acquisition unit 41 may perform communication with the input device 100 via the communication unit 27, and acquire a measurement value of the proximity sensor of the input device 100. The state information acquisition unit 41 may determine whether the input device 100 is installed around the main body portion 2 based on the measurement value of the proximity sensor of the input device 100. In a case in which the measurement value of the proximity sensor of the input device 100 is less than the threshold, the state information acquisition unit 41 determines that the input device 100 is installed around the main body portion 2. For example, in a case in which the input device 100 is installed on a shelf, the distance between the input device 100 and the shelf is short, and therefore the measurement value of the proximity sensor of the input device 100 is less than the threshold. In a case in which the measurement value of the proximity sensor of the input device 100 is the threshold or more, the state information acquisition unit 41 determines that the input device 100 is not installed around the main body portion 2. In a case in which the input device 100 is not installed around the main body portion 2, the distance between the input device 100 and the object is long, and therefore the measurement value of the proximity sensor of the input device 100 is the threshold or more.

The state information of the input device 100 may include information indicating whether the subject is gripping the input device 100. The process of determining whether the input device 100 is being gripped by the subject will be described. The state information acquisition unit 41 may perform communication with the input device 100 via the communication unit 27, and acquire acceleration information (measurement value of the acceleration sensor) of the input device 100. The state information acquisition unit 41 may determine whether the input device 100 is being gripped by the subject based on the measurement value of the acceleration sensor of the input device 100. In a case in which the measurement value of the acceleration sensor of the input device 100 is a threshold or more, the state information acquisition unit 41 determines that the input device 100 is being gripped by the subject. In this case, the state information of the input device 100 includes information indicating that the subject is gripping the input device 100. In a case in which the subject is gripping the input device 100, the input device 100 is moving, and the measurement value of the acceleration sensor of the input device 100 is the threshold or more. In a case in which the measurement value of the acceleration sensor of the input device 100 is less than the threshold, the state information acquisition unit 41 determines that the input device 100 is not being gripped by the subject. In this case, the state information of the input device 100 includes information indicating that the subject is not gripping the input device 100. In a case in which the subject is not gripping the input device 100, the input device 100 is not moving, and therefore the measurement value of the acceleration sensor of the input device 100 is less than the threshold.

In step S103, the biological information measurement unit 43 selects a measurement sequence corresponding to the state information of the input device 100 to measure the biological information of the subject on the front surface of the main body portion 2. The biological information measurement unit 43 may select at least one measurement item of a plurality of measurement items related to the living body of the subject on the front surface of the main body portion 2 based on the state information of the input device 100 to measure the biological information of the subject. The plurality of measurement items related to the living body of the subject may include a measurement item for measuring the weight, a measurement item for measuring the body composition, a measurement item for measuring the standing balance, and a measurement item for measuring the cardiovascular state.

The biological information measurement unit 43 selects a measurement sequence corresponding to information indicating that the input device 100 is installed on the main body portion 2 or information indicating that the input device 100 is not installed on the main body portion 2 to measure the biological information of the subject on the front surface of the main body portion 2. This enables the biological information of the subject to be measured in consideration of the state of the input device 100 that causes body motion. In a case in which the state information of the input device 100 includes information indicating that the input device 100 is installed on the main body portion 2, the biological information measurement unit 43 may select the measurement sequence for measuring the weight, the body composition, the standing balance, and the cardiovascular state to measure the biological information of the subject. By installing the input device 100 in the main body portion 2, body motion caused by the subject gripping the input device 100 is suppressed. In a case in which the measurement sequence corresponding to the information indicating that the input device 100 is installed on the main body portion 2 is selected, measurement of the biological information of the subject is performed in a state in which body motion is suppressed, and therefore the measurement accuracy is improved. The biological information measurement unit 43 may select the measurement item for measuring the weight, the body composition, the standing balance, and the cardiovascular state of the plurality of measurement items related to the living body of the subject to measure the biological information of the subject. In a case in which the input device 100 is installed on the main body portion 2, measurement of the biological information of the subject is performed on an assumption that the subject intends to have the weight, the body composition, the standing balance, and the cardiovascular state measured. In the case in which the state information of the input device 100 includes information indicating that the input device 100 is installed on the main body portion 2, the biological information measurement unit 43 may select all measurement items of the plurality of measurement items related to the living body of the subject to measure the biological information of the subject.

In a case in which the state information of the input device 100 includes information indicating that the input device 100 is not installed on the main body portion 2, the biological information measurement unit 43 may select at least one of the measurement sequence for measuring the weight and the measurement sequence for measuring the body composition to measure the biological information of the subject. Measurement of the weight and the body composition of the subject is not affected by body motion of the subject. In a case in which the input device 100 is not installed on the main body portion 2, there is a possibility that body motion occurs by the subject gripping the input device 100 or the subject operating the input device 100. In a case in which the measurement sequence corresponding to the information indicating that the input device 100 is not installed on the main body portion 2 is selected, remeasurement due to the body motion can be prevented, and therefore the measurement success rate is improved. The biological information measurement unit 43 may select at least one of the measurement item for measuring the weight and the measurement item for measuring the body composition of the plurality of measurement items related to the living body of the subject to measure biological information of the subject. For example, in a case in which the state of the input device 100 is a state that leads to the occurrence of body motion, the measurement success rate is improved by selecting a measurement item hardly affected by the body motion. In a case in which a measurement item not easily affected by body motion is selected, no other measurement item is selected, and therefore the measurement time is shortened. The number of measurement items selected in the case in which the state information of the input device 100 includes the information indicating that the input device 100 is not installed on the main body portion 2 is smaller than the number of measurement items selected in the case in which the state information of the input device 100 includes information indicating that the input device 100 is installed on the main body portion 2. At least one of the weight and the body composition of the subject is measured and other measurement items are not measured, and therefore the time from the start to the completion of measurement of the biological information of the subject is shortened.

The biological information measurement unit 43 selects a measurement sequence corresponding to information indicating that the input device 100 is installed around the main body portion 2 or information indicating that the input device 100 is not installed around the main body portion 2 to measure the biological information of the subject on the front surface of the main body portion 2. This enables the biological information of the subject to be measured in consideration of the state of the input device 100 that causes body motion. In a case in which the state information of the input device 100 includes information indicating that the input device 100 is installed around the main body portion 2, the biological information measurement unit 43 may select the measurement sequence for measuring the weight, the body composition, the standing balance, and the cardiovascular state to measure the biological information of the subject. By installing the input device 100 around the main body portion 2, body motion caused by the subject gripping the input device 100 is suppressed. In a case in which the measurement sequence corresponding to the information indicating that the input device 100 is installed on the main body portion 2 is selected, measurement of the biological information of the subject is performed in a state in which body motion is suppressed, and therefore the measurement accuracy is improved. The biological information measurement unit 43 may select the measurement item for measuring the weight, the body composition, the standing balance, and the cardiovascular state of the plurality of measurement items related to the living body of the subject to measure the biological information of the subject. In a case in which the input device 100 is installed around the main body portion 2, measurement of the biological information of the subject is performed on an assumption that the subject intends to have the weight, the body composition, the standing balance, and the cardiovascular state measured. In the case in which the state information of the input device 100 includes information indicating that the input device 100 is installed around the main body portion 2, the biological information measurement unit 43 may select all measurement items of the plurality of measurement items related to the living body of the subject to measure the biological information of the subject.

In a case in which the state information of the input device 100 includes information indicating that the input device 100 is not installed around the main body portion 2, the biological information measurement unit 43 may select at least one of the measurement sequence for measuring the weight and the measurement sequence for measuring the body composition to measure the biological information of the subject. Measurement of the weight and the body composition of the subject is not affected by body motion of the subject. In a case in which the input device 100 is not installed around the main body portion 2, there is a possibility that body motion occurs by the subject gripping the input device 100 or the subject operating the input device 100. In a case in which the measurement sequence corresponding to the information indicating that the input device 100 is not installed around the main body portion 2 is selected, remeasurement due to the body motion can be prevented, and therefore the measurement success rate is improved. The biological information measurement unit 43 may select at least one of the measurement item for measuring the weight and the measurement item for measuring the body composition of the plurality of measurement items related to the living body of the subject to measure biological information of the subject. For example, in a case in which the state of the input device 100 is a state that leads to the occurrence of body motion, the measurement success rate is improved by selecting a measurement item hardly affected by the body motion. In a case in which a measurement item not easily affected by body motion is selected, no other measurement item is selected, and therefore the measurement time is shortened. The number of measurement items selected in the case in which the state information of the input device 100 includes the information indicating that the input device 100 is not installed around the main body portion 2 is smaller than the number of measurement items selected in the case in which the state information of the input device 100 includes information indicating that the input device 100 is installed around the main body portion 2. At least one of the weight and the body composition of the subject is measured and other measurement items are not measured, and therefore the time from the start to the completion of measurement of the biological information of the subject is shortened.

The biological information measurement unit 43 selects a measurement sequence corresponding to information indicating that the subject is gripping the input device 100 or information indicating that the subject is not gripping the input device 100 to measure the biological information of the subject on the front surface of the main body portion 2. This enables the biological information of the subject to be measured in consideration of the state of the input device 100 that causes body motion. In a case in which the state information of the input device 100 includes information indicating that the subject is not gripping the input device 100, the biological information measurement unit 43 may select the measurement sequence for measuring the weight, the body composition, the standing balance, and the cardiovascular state to measure the biological information of the subject. The biological information measurement unit 43 may select the measurement item for measuring the weight, the body composition, the standing balance, and the cardiovascular state of the plurality of measurement items related to the living body of the subject to measure the biological information of the subject. In a case in which the subject is not gripping the input device 100 measurement of the biological information of the subject is performed on an assumption that the subject intends to have the weight, the body composition, the standing balance, and the cardiovascular state measured. In a case in which the state information of the input device 100 includes information indicating that the subject is not gripping the input device 100, the biological information measurement unit 43 may select all measurement items of the plurality of measurement items related to the living body of the subject to measure the biological information of the subject.

In a case in which the state information of the input device 100 includes information indicating that the subject is gripping the input device 100, the biological information measurement unit 43 may select at least one of the measurement sequence for measuring the weight and the measurement sequence for measuring the body composition to measure the biological information of the subject. Measurement of the weight and the body composition of the subject is not affected by body motion of the subject. In a case in which the subject is gripping the input device 100, there is a possibility that body motion occurs by the subject operating the input device 100. In a case in which the measurement sequence corresponding to the information indicating that the subject is gripping the input device 100 is selected, remeasurement due to the body motion can be prevented, and therefore the measurement success rate is improved. The biological information measurement unit 43 may select at least one of the measurement item for measuring the weight and the measurement item for measuring the body composition of the plurality of measurement items related to the living body of the subject to measure biological information of the subject. For example, in a case in which the state of the input device 100 is a state that leads to the occurrence of body motion, the measurement success rate is improved by selecting a measurement item hardly affected by the body motion. In a case in which a measurement item not easily affected by body motion is selected, no other measurement item is selected, and therefore the measurement time is shortened. The number of measurement items selected in the case in which the state information of the input device 100 includes the information indicating that the subject is gripping the input device 100 is smaller than the number of measurement items selected in the case in which the state information of the input device 100 includes information indicating that the subject is not gripping the input device 100. At least one of the weight and the body composition of the subject is measured and other measurement items are not measured, and therefore the time from the start to the completion of measurement of the biological information of the subject is shortened.

In step S102, the body motion measurement unit 42 may measure the body motion of the subject on the front surface of the main body portion 2 based on the measurement value output from at least one of the first sensor unit 23, the second sensor unit 24, and the electrode unit 21, and determine whether the body motion of the subject is large. That is, the body motion measurement unit 42 may measure the body motion of the subject on the front surface of the biological information measurement device 1 to determine whether a stationary state of the subject is continuing. The stationary state is a state in which there is no fluctuation of a predetermined value or more regarding the measurement value output from the first sensor unit 23, the second sensor unit 24, or the electrode unit 21. The measurement values output from the load cells 31 to 34 may be affected by vibration of the input device 100. Therefore, the body motion measurement unit 42 may measure the body motion of the subject on the front surface of the main body portion 2 based on the measurement value output from the acceleration sensor 35 without using the measurement values output from the load cells 31 to 34.

The process of determining whether the body motion of the subject is large will be described. In a case in which the foot of the subject on the front surface of the main body portion 2 moves, the measurement value output from the first sensor unit 23 fluctuates. The measurement value output from the first sensor unit 23 is, for example, a voltage value or a light reception amount of the photodiode. In a case in which the measurement value output from the first sensor unit 23 fluctuates continuously for a predetermined time, the body motion measurement unit 42 determines that the body motion of the subject is large. In a case in which the measurement value output from the first sensor unit 23 fluctuates continuously for the predetermined time, the body motion measurement unit 42 may determine that the stationary state of the subject is not continuing. The body motion measurement unit 42 outputs a measurement result including a result indicating that the stationary state of the subject is not continuing (result indicating that the body motion of the subject is large). In a case in which the measurement value output from the first sensor unit 23 does not fluctuate continuously for the predetermined time, the body motion measurement unit 42 determines that the body motion of the subject is small. In a case in which the measurement value output from the first sensor unit 23 does not fluctuate continuously for the predetermined time, the body motion measurement unit 42 may determine that the stationary state of the subject is continuing. The body motion measurement unit 42 outputs a measurement result including a result indicating that the stationary state of the subject is continuing (result indicating that the body motion of the subject is small).

In a case in which the subject on the front surface of the main body portion 2 performs a predetermined motion, the measurement value output from the second sensor unit 24 increases. For example, the predetermined motion is, but is not limited to, the subject moving their head. In a case in which the measurement value output from the second sensor unit 24 is a threshold or more continuously for a predetermined time, the body motion measurement unit 42 determines that the body motion of the subject is large. In a case in which the mean value of the measurement values output from the second sensor unit 24 within a predetermined time is the threshold or more, the body motion measurement unit 42 determines that the body motion of the subject is large. In these cases, the body motion measurement unit 42 may determine that the stationary state of the subject is not continuing. The body motion measurement unit 42 outputs a measurement result including a result indicating that the stationary state of the subject is not continuing (result indicating that the body motion of the subject is large). In a case in which the measurement value output from the second sensor unit 24 is less than the threshold continuously for the predetermined time, the body motion measurement unit 42 determines that the body motion of the subject is small. In a case in which the mean value of the measurement values output from the second sensor unit 24 within the predetermined time is less than the threshold, the body motion measurement unit 42 determines that the body motion of the subject is small. In these cases, the body motion measurement unit 42 may determine that the stationary state of the subject is continuing. The body motion measurement unit 42 outputs a measurement result including a result indicating that the stationary state of the subject is continuing (result indicating that the body motion of the subject is small). The threshold used for measuring the body motion of the subject may be obtained by an experiment or a simulation. For example, in a case in which the subject performs a motion of viewing the display unit 3, the threshold may be determined based on the measurement value output from the second sensor unit 24.

In a case in which the foot of the subject on the front surface of the main body portion 2 moves, the measurement value output from the electrode unit 21 fluctuates. The measurement value output from the electrode unit 21 is, for example, a myoelectric. In a case in which the measurement value output from the electrode unit 21 fluctuates continuously for a predetermined time, the body motion measurement unit 42 determines that the body motion of the subject is large. In a case in which the measurement value output from the electrode unit 21 fluctuates continuously for the predetermined time, the body motion measurement unit 42 may determine that the stationary state of the subject is not continuing. The body motion measurement unit 42 outputs a measurement result including a result indicating that the stationary state of the subject is not continuing (result indicating that the body motion of the subject is large). In a case in which the measurement value output from the electrode unit 21 does not fluctuate continuously for the predetermined time, the body motion measurement unit 42 determines that the body motion of the subject is small. In a case in which the measurement value output from the electrode unit 21 does not fluctuate continuously for the predetermined time, the body motion measurement unit 42 may determine that the stationary state of the subject is continuing. The body motion measurement unit 42 outputs a measurement result including a result indicating that the stationary state of the subject is continuing (result indicating that the body motion of the subject is small).

In step S103, the biological information measurement unit 43 may select a measurement sequence corresponding to the state information of the input device 100 and the measurement result of the body motion measurement unit 42 to measure the biological information of the subject. For example, even in a case in which the state of the input device 100 is a state that leads to the occurrence of the body motion of the subject, there is a case in which the body motion of the subject does not occur. For example, even in a case in which the state of the input device 100 is a state that does not lead to the occurrence of the body motion of the subject, there is a case in which the body motion of the subject occurs. By the body motion measurement unit 42 measuring the body motion of the subject, the state of the subject can be captured more accurately, and the biological information of the subject can be measured more accurately. The biological information measurement unit 43 may select at least one measurement item of the plurality of measurement items related to the living body of the subject on the front surface of the main body portion 2 based on the state information of the input device 100 and the measurement result of the body motion measurement unit 42 to measure the biological information of the subject. For example, in a case in which the state of the input device 100 does not lead to the occurrence of body motion but body motion of the subject occurs, the measurement success rate is improved by selecting a measurement item hardly affected by the body motion. In a case in which a measurement item not easily affected by body motion is selected, no other measurement item is selected, and therefore the measurement time is shortened.

The case in which the state information of the input device 100 includes information indicating that the input device 100 is installed on the main body portion 2, and a case in which the measurement result of the body motion measurement unit 42 includes a result indicating that the stationary state of the subject is continuing will be described. The biological information measurement unit 43 may select the measurement sequence for measuring the weight, the body composition, the standing balance, and the cardiovascular state to measure the biological information of the subject. The biological information measurement unit 43 may select the measurement item for measuring the weight, the body composition, the standing balance, and the cardiovascular state of the plurality of measurement items related to the living body of the subject to measure the biological information of the subject. Alternatively, the biological information measurement unit 43 may select all measurement items of the plurality of measurement items related to the living body of the subject to measure the biological information of the subject.

A case in which the state information of the input device 100 includes information indicating that the input device 100 is not installed on the main body portion 2, or a case in which the measurement result of the body motion measurement unit 42 includes a result indicating that the stationary state of the subject is not continuing will be described. The biological information measurement unit 43 may select at least one of the measurement sequence for measuring the weight and the measurement sequence for measuring the body composition to measure the biological information of the subject. The biological information measurement unit 43 may select at least one of the measurement item for measuring the weight and the measurement item for measuring the body composition of the plurality of measurement items related to the living body of the subject to measure biological information of the subject.

The case in which the state information of the input device 100 includes information indicating that the input device 100 is installed around the main body portion 2, and a case in which the measurement result of the body motion measurement unit 42 includes a result indicating that the stationary state of the subject is continuing will be described. The biological information measurement unit 43 may select the measurement sequence for measuring the weight, the body composition, the standing balance, and the cardiovascular state to measure the biological information of the subject. The biological information measurement unit 43 may select the measurement item for measuring the weight, the body composition, the standing balance, and the cardiovascular state of the plurality of measurement items related to the living body of the subject to measure the biological information of the subject. Alternatively, the biological information measurement unit 43 may select all measurement items of the plurality of measurement items related to the living body of the subject to measure the biological information of the subject.

A case in which the state information of the input device 100 includes information indicating that the input device 100 is not installed around the main body portion 2, or a case in which the measurement result of the body motion measurement unit 42 includes a result indicating that the stationary state of the subject is not continuing will be described. The biological information measurement unit 43 may select at least one of the measurement sequence for measuring the weight and the measurement sequence for measuring the body composition to measure the biological information of the subject. The biological information measurement unit 43 may select at least one of the measurement item for measuring the weight and the measurement item for measuring the body composition of the plurality of measurement items related to the living body of the subject to measure biological information of the subject.

The case in which the state information of the input device 100 includes information indicating that the subject is not gripping the input device 100, and a case in which the measurement result of the body motion measurement unit 42 includes a result indicating that the stationary state of the subject is continuing will be described. The biological information measurement unit 43 may select the measurement sequence for measuring the weight, the body composition, the standing balance, and the cardiovascular state to measure the biological information of the subject. The biological information measurement unit 43 may select the measurement item for measuring the weight, the body composition, the standing balance, and the cardiovascular state of the plurality of measurement items related to the living body of the subject to measure the biological information of the subject. Alternatively, the biological information measurement unit 43 may select all measurement items of the plurality of measurement items related to the living body of the subject to measure the biological information of the subject.

A case in which the state information of the input device 100 includes information indicating that the subject is gripping the input device 100, or a case in which the measurement result of the body motion measurement unit 42 includes a result indicating that the stationary state of the subject is not continuing will be described. The biological information measurement unit 43 may select at least one of the measurement sequence for measuring the weight and the measurement sequence for measuring the body composition to measure the biological information of the subject. The biological information measurement unit 43 may select at least one of the measurement item for measuring the weight and the measurement item for measuring the body composition of the plurality of measurement items related to the living body of the subject to measure biological information of the subject.

A guide display that prompts installation of the input device 100 may be displayed on the display unit of the input device 100. The guide display may be a message that prompts installation of the input device 100 on the main body portion 2. The guide display may be a message that prompts installation of the input device 100 around the main body portion 2. The sound output device of the input device 100 may output a guide sound that prompts installation of the input device 100. The guide sound may be a sound that prompts installation of the input device 100 on the main body portion 2. The guide sound may be a sound that prompts installation of the input device 100 around the main body portion 2. A process of guide display and a process of guide sound may be performed before the subject steps on the front surface of the main body portion 2. In a case in which the subject is gripping the input device 100, the process of guide display and the process of guide sound may be performed.

In a case in which the state information of the input device 100 includes information indicating that the subject is gripping the input device 100, the state information acquisition unit 41 may perform communication with the input device 100 via the communication unit 27, and issue a predetermined instruction to the input device 100. The input device 100 displays a guidance display on the display unit of the input device 100 based on a predetermined instruction. The guidance display may be performed by an application of the input device 100. The guidance display may be a display that prompts the subject to place a finger on the screen of the display unit of the input device 100. The guidance display may be a display using a level application. The guidance display may be a display indicating a remaining time until completion of the measurement of the biological information. Since the subject places the finger on the screen of the display unit of the input device 100, the subject cannot operate the input device 100 while the measurement of the biological information is being performed. Due to the guidance display being displayed on the display unit of the input device 100, the subject gazes at the display unit of the input device 100. A motion in which the subject moves their head or the like to look to their feet is suppressed, and the body motion of the subject can be suppressed. This improves the measurement accuracy of the biological information of the subject.

For example, a case will be described in which a sound or a notification sound is output from an information processing terminal that is an example of the input device 100 and the subject shifts to a state of gripping the information processing terminal while measurement of the biological information regarding all measurement items including items related to the cardiovascular state greatly affected by body motion is being performed in a state in which the information processing terminal is placed in a peripheral portion such as a wash basin. In this case, a message to interrupt the measurement of the biological information is displayed on the display unit 3, and the biological information regarding a measurement item for which measurement has been completed is stored in the storage unit 28. The biological information regarding the measurement item for which measurement has been completed may be displayed on the display unit 3. A measurement item for which measurement has not been completed may be displayed on the display unit 3. The measurement item for which measurement has been completed may be displayed on the display unit of the information processing terminal. The measurement item for which measurement has not been completed may be displayed on the display unit of the information processing terminal. For example, a message including necessity of remeasurement, recording of measurement data, and remaining time until completion of measurement may be displayed on the display unit 3 or may be displayed on the display unit of the information processing terminal. In a case in which the body motion of the subject occurs due to the subject gripping the information processing device and the measurement time of the biological information is prolonged, a message indicating that the measurement time has been prolonged may be displayed on the display unit 3. By the subject recognizing the cause of the prolonged measurement time, the body motion of the subject in the measurement of the biological information of the next time is suppressed. In this manner, even in a case in which a state change of the input device 100 occurs in the middle of measurement, the measurement sequence is changed and selected in accordance with state information of the input device 100, body motion suppression notification is given to the subject, or the measurement item is switched to a measurement item hardly affected by body motion at an early stage. Therefore, measurement is easily completed, which leads to an improvement in the measurement success rate. Therefore, it is possible to realize desirable measurement for the subject.

In a case in which a sound is output from the information processing terminal and the subject is gripping the information processing terminal, the biological information measurement unit 43 excludes each data while measurement of the biological information is being performed. In this case, the biological information measurement unit 43 performs a process of extending measurement of the biological information. The display unit 3 may display that the process of extending the measurement of the biological information has been performed by the subject gripping the information processing terminal. By the subject recognizing that the process of extending the measurement of the biological information is performed, the body motion of the subject in the measurement of the biological information of the next time is suppressed. For example, in a case in which a dedicated application is incorporated in the information processing terminal and the dedicated application is activated, a sound may not be output from the information processing terminal while measurement of the biological information is being performed.

The biological information measurement device 1 may include a sound output device. In a case in which the input device 100 is in a state of being installed on the main body portion 2 even a predetermined time has elapsed since measurement of the biological information is completed, the sound output device of the biological information measurement device 1 may output an alarm sound. This can suppress the state in which the input device 100 is installed on the main body portion 2 from being left.

In a case in which measurement of the biological information of the subject is being performed for all the measurement items of the plurality of measurement items related to the living body of the subject, the display unit 3 may not display information while the biological information measurement unit 43 is performing the measurement process. A motion in which the subject views information displayed on the display unit 3 (motion of looking to their feet) affects measurement of the cardiovascular state. By the display unit 3 not displaying information while the biological information measurement unit 43 is performing the measurement process, the body motion of the subject can be suppressed, and the accuracy in measuring the cardiovascular state is improved.

### Computer Readable Recording Medium

A program for causing an information processing device, other machines and apparatuses (hereinafter, referred to as a computer or the like) to realize any of the functions described above can be recorded on a recording medium readable by the computer and the like. Then, the computer or the like is caused to load the program onto a recording medium and execute the program, thereby providing the functions.

Here, the recording medium readable by the computer or the like refers to a recording medium that can store information such as data and programs by an electrical, magnetic, optical, mechanical, or chemical action and can be read from the computer or the like. Among such recording media, those removable from the computer or the like include, for example, a flexible disk, a magneto-optical disk, a CD-ROM, a CD-R/W, a DVD, a Blu-ray disk, a flash memory, and the like. Further, such a recording medium fixed to the computer or the like includes a hard disk, a ROM, and the like.

### REFERENCE SIGNS LIST

1: Biological information measurement device
2: Main body portion
3: Display unit
4, 5: Current applying electrode
6, 7: Voltage measuring electrode
20: Control unit
21: Electrode unit
22: Impedance measurement unit
23: First sensor unit
24: Second sensor unit
25: Third sensor unit
26: Temperature measurement unit
27: Communication unit
28: Storage unit
31, 32, 33, 34: Load cell
35: Acceleration sensor
41: State information acquisition unit
42: Body motion measurement unit
43: Biological information measurement unit
51: Weight measurement unit
52: Body composition measurement unit
53: Standing balance measurement unit
54: Cardiovascular measurement unit
100: Input device

What is claimed is:

1. A biological information measurement device comprising:
a main body portion including a placement surface on which a subject can step;
a state information acquisition unit that acquires state information of an input device operated by the subject; and
a biological information measurement unit that selects a measurement sequence corresponding to the state information to measure biological information of the subject on the placement surface, wherein
the biological information measurement unit selects at least one measurement item of a plurality of measurement items related to a living body of the subject on the placement surface based on the state information to measure the biological information of the subject,
the state information includes information indicating whether the subject is gripping the input device, and
the number of measurement items selected in a case in which the state information includes information indicating that the subject is gripping the input device is smaller than the number of measurement items selected in a case in which the state information includes information indicating that the subject is not gripping the input device.

2. The biological information measurement device according to claim 1, wherein
the state information includes information indicating whether the input device is installed on the main body portion, and
the biological information measurement unit selects the measurement sequence corresponding to information indicating that the input device is installed on the main body portion or information indicating that the input device is not installed on the main body portion to measure the biological information of the subject.

3. The biological information measurement device according to claim 1, wherein
the state information includes information indicating whether the input device is installed around the main body portion, and
the biological information measurement unit selects the measurement sequence corresponding to information indicating that the input device is installed around the main body portion or information indicating that the input device is not installed around the main body portion to measure the biological information of the subject.

4. The biological information measurement device according to claim 1, wherein
the state information includes information indicating whether the subject is gripping the input device, and
the biological information measurement unit selects the measurement sequence corresponding to information indicating that the subject is gripping the input device or information indicating that the subject is not gripping the input device to measure the biological information of the subject.

5. The biological information measurement device according to claim 1 comprising
a body motion measurement unit that measures body motion of the subject on the placement surface, wherein
the biological information measurement unit selects the measurement sequence corresponding to the state information and a measurement result of the body motion measurement unit to measure the biological information of the subject on the placement surface.

6. The biological information measurement device according to claim 2 comprising
a body motion measurement unit that measures body motion of the subject on the placement surface, wherein
the biological information measurement unit selects the measurement sequence corresponding to the state information and a measurement result of the body motion measurement unit to measure the biological information of the subject on the placement surface.

7. The biological information measurement device according to claim 3 comprising
a body motion measurement unit that measures body motion of the subject on the placement surface, wherein
the biological information measurement unit selects the measurement sequence corresponding to the state information and a measurement result of the body motion measurement unit to measure the biological information of the subject on the placement surface.

8. The biological information measurement device according to claim 4 comprising
a body motion measurement unit that measures body motion of the subject on the placement surface, wherein
the biological information measurement unit selects the measurement sequence corresponding to the state information and a measurement result of the body motion measurement unit to measure the biological information of the subject on the placement surface.

9. The biological information measurement device according to claim 1 comprising
a body motion measurement unit that measures body motion of the subject on the placement surface, wherein
the biological information measurement unit selects at least one measurement item of the plurality of measurement items related to the living body of the subject based on the state information and a measurement result of the body motion measurement unit to measure the biological information of the subject on the placement surface.

10. A biological information measurement method, wherein
a computer of a biological information measurement device including a main body portion including a placement surface on which a subject can step executes
a state information acquisition step of acquiring state information of an input device operated by the subject, and
a biological information measurement step of selecting a measurement sequence corresponding to the state information to measure biological information of the subject on the placement surface,
in the biological information measurement step, at least one measurement item of a plurality of measurement items related to a living body of the subject on the placement surface is selected based on the state information to measure the biological information of the subject,
the state information includes information indicating whether the subject is gripping the input device, and
the number of measurement items selected in a case in which the state information includes information indicating that the subject is gripping the input device is smaller than the number of measurement items selected in a case in which the state information includes information indicating that the subject is not gripping the input device.

11. A recording medium for causing
a computer of a biological information measurement device including a main body portion including a placement surface on which a subject can step to execute
a state information acquisition step of acquiring state information of an input device operated by the subject, and
a biological information measurement step of selecting a measurement sequence corresponding to the state information to measure biological information of the subject on the placement surface, wherein
in the biological information measurement step, at least one measurement item of a plurality of measurement items related to a living body of the subject on the placement surface is selected based on the state information to measure the biological information of the subject,
the state information includes information indicating whether the subject is gripping the input device, and
the number of measurement items selected in a case in which the state information includes information indicating that the subject is gripping the input device is smaller than the number of measurement items selected in a case in which the state information includes information indicating that the subject is not gripping the input device.
